(19) United States
(12) Patent Application Publication
(10) Pub. No.: US 2026/0176585 A1
(43) Pub. Date: Jun. 25, 2026

(54) CULTIVATION METHOD FOR ENHANCING DIFFERENTIATION EFFICIENCY AND FUNCTIONALITY OF NK CELLS

(71) Applicant: MARU THERAPEUTICS CO., LTD., Seoul (KR)

(72) Inventors: Kyung-Sun KANG, Seoul (KR); Daekee KWON, Seoul (KR); Bo Kyung MOON, Seoul (KR)

(73) Assignee: MARU THERAPEUTICS CO., LTD., Seoul (KR)

(21) Appl. No.: 19/127,240

(22) PCT Filed: Nov. 7, 2023
(86) PCT No.: PCT/KR2023/017797

§ 371 Date: May 5, 2025

(30) Foreign Application Priority Data
Nov. 7, 2022 (KR) 10-2022-0146970
Nov. 7, 2023 (KR) 10-2023-0153063

(51) Int. Cl. C12N 5/0783; C12N 5/00

(52) U.S. Cl. CPC C12N 5/0646; C12N 5/0018; C12N 2500/12; C12N 2500/38; C12N 2500/44; C12N 2500/46; C12N 2501/125; C12N 2501/2303; C12N 2501/2307; C12N 2501/2315; C12N 2501/26; C12N 2501/727; C12N 2506/11

(57) ABSTRACT

Provided is a cultivation method for enhancing the differentiation efficiency and functionality of NK cells, based on the finding that the differentiation efficiency of NK cells is increased by selecting and differentiating cells exhibiting specific characteristics, wherein the efficiency of NK cell differentiation from induced pluripotent stem cells was enhanced by altering conditions at specific stages of the process of differentiating induced pluripotent cells into NK cells, conditions for the composition of stage-specific media used, and the treatment methods for the composition, and in the light of acquiring functional NK cells as well as such quantitative increase, the method can be advantageously used for qualitative improvement.

### TECHNICAL FIELD

The present invention relates to a culture method for increasing the differentiation efficiency and functionality of NK cells.

This application claims priority to and the benefit of Korean Patent Application No. 10-2022-0146970, filed on Nov. 7, 2022, the disclosure of which is incorporated herein by reference in its entirety.

This application claims priority to and the benefit of Korean Patent Application No. 10-2023-0153063, filed on Nov. 7, 2023, the disclosure of which is incorporated herein by reference in its entirety.

### BACKGROUND ART

Natural killer cells (hereinafter referred to as “NK cells”), which are part of the immune system, recognize abnormally transformed cell membranes, as in cells infected with viruses or cancer cells. NK cells play an essential role in the early stages of viral infection or tumor formation before large numbers of activated cytotoxic T lymphocytes are produced. Histologically, NK cells are large granular lymphocytes. The intracellular granules contain preformed molecules with potent biological functions, and the molecules are released when NK cells come into contact with target cells. Some of the molecules form holes in the membrane of target cells, causing cell lysis, or invade target cells and increase the fragmentation of nuclear DNA, causing apoptosis or programmed cell death.

NK cells are known to be cells that can kill cancer nonspecifically. The killing ability of these NK cells is being applied as a new cell therapy to treat solid tumors using lymphokine-activated killer cells (LAK) and tumor-infiltrating lymphocytes (TIL), or to prevent rejection reactions that occur during bone marrow transplantation or organ transplantation by performing immunotherapy through donor lymphocyte infusion.

In addition, defects in the differentiation and activity of NK cells have been reported to be associated with various cancers, including breast cancer, melanoma, and lung cancer. To treat these diseases, NK cell therapy is emerging.

To effectively use NK cells as anticancer immunotherapy agents, securing a large number of NK cells is necessary. However, NK cells account for 10 to 15% of lymphocytes in the blood, and in cancer patients, the number, differentiation, and function of NK cells are often reduced, making it difficult to secure a sufficient number of cells. Therefore, to apply NK cells as an NK cell therapy, its mass production through its proliferation or differentiation is required.

### DISCLOSURE

### Technical Problem

One object of the present invention is to provide a method for differentiating NK cells from stem cells, the method including a step of selecting hematopoietic progenitor cells expressing APC (Allophycocyanin) among hematopoietic progenitor cells cultured from stem cells.

Another object of the present invention is to provide a method for differentiating NK cells from stem cells, the method including a step of culturing and differentiating NK cells from hematopoietic progenitor cells in a first NK cell differentiation medium or a second NK cell differentiation medium including at least one selected from the group consisting of:

β-mercaptoethanol (BME, 2BME, 2-ME, or β-met), sodium selenite, ethanolamine, ascorbic acid, and IL-3.

Still another object of the present invention is to provide a medium composition for differentiating NK cells from stem cells, the composition including, as an active ingredient, at least one selected from the group consisting of:

β-mercaptoethanol (BME, 2BME, 2-ME, or β-met), sodium selenite, ethanolamine, ascorbic acid, IL-3, and adezmapimod (SB203580).

Still another object of the present invention is to provide a kit for differentiating stem cells into NK cells, the kit including the composition and an instructions.

However, technical problems to be solved in the present invention are not limited to the above-described problems, and other problems which are not described herein will be fully understood by those of ordinary skill in the art from the following descriptions.

### Technical Solution

The present invention provides a method for differentiating NK cells from stem cells, the method including a step of selecting Allophycocyanin (APC)-expressing hematopoietic progenitor cells among hematopoietic progenitor cells cultured from stem cells.

In one embodiment of the present invention, the hematopoietic progenitor cells may additionally express one or more markers selected from the group consisting of CD34, CD31, CD43, and CD45, but are not limited thereto.

In one embodiment of the present invention, the differentiation efficiency of NK cells differentiated from the hematopoietic progenitor cells may be increased, but is not limited thereto.

In one embodiment of the present invention, the NK cells differentiated from the hematopoietic progenitor cells may have increased cytotoxic function, but are not limited thereto.

The present invention provides a method for differentiating NK cells from stem cells, the method including: culturing hematopoietic progenitor cells and differentiating NK cells from the hematopoietic progenitor cells in a first NK cell differentiation medium or second NK cell differentiation medium including at least one selected from the group consisting of:
β-mercaptoethanol (BME, 2BME, 2-ME or β-met), sodium selenite, ethanolamine, ascorbic acid, and IL-3.

In one embodiment of the present invention, the second NK cell differentiation medium may not include IL-3, but is not limited thereto.

In one embodiment of the present invention, the first NK cell differentiation medium or the second NK cell differentiation medium may include, based on the total composition, 0.1 to 20 μM of β-mercaptoethanol, 0.1 to 50 ng/ml of sodium selenite, 1 to 600 μM of ethanolamine, and 1 to 200 mg/L of ascorbic acid, but is not limited thereto.

In one embodiment of the present invention, the first NK cell differentiation medium or the second NK cell differentiation medium may further include one or more selected from the group consisting of stemline, stempro-34, SCF, IL-7, IL-15, and FLT3 ligand, but is not limited thereto.

In one embodiment of the present invention, the NK cells may have increased expression of an activating receptor, but are not limited thereto.

In one embodiment of the present invention, the activating receptor may be one or more selected from CD16 or NKG2D, but is not limited thereto.

In one embodiment of the present invention, the second NK cell differentiation medium may further include a p38 MAPK inhibitor, but is not limited thereto.

In one embodiment of the present invention, the p38 MAPK inhibitor may be one or more selected from the group consisting of adezmapimod (SB203580), losmapimod, doramapimod, ralimetinib, and neflamapimod (VX-745), but is not limited thereto.

In one embodiment of the present invention, the NK cells may have reduced expression of an inhibitory receptors, but are not limited thereto.

In one embodiment of the present invention, the inhibitory receptor may be NKG2A, but is not limited thereto.

In one embodiment of the present invention, the NK cells may maintain or increase the expression level of one or more activating receptors selected from the group consisting of CD16, NKG2D, NKp30, and NKp44, but are not limited thereto.

In one embodiment of the present invention, the culturing performed in the first NK cell differentiation medium or the second NK cell differentiation medium may be performed from day 10 to 14 to day 52 to 56 of the entire incubation process, but is not limited thereto.

In one embodiment of the present invention, the culturing performed in the first NK cell differentiation medium may be performed from day 10 to 14 to day 16 to 20 of entire incubation process, but is not limited thereto.

In one embodiment of the present invention, the culturing performed in the second NK cell differentiation medium may be performed from day 16 to 20 to day 46 to 52 of entire incubation process, but is not limited thereto.

In one embodiment of the present invention, the NK cells may exhibit increased cytotoxicity, but are not limited thereto.

In one embodiment of the present invention, the stem cell may be one or more selected from the group consisting of induced pluripotent stem cells, embryonic stem cells, and adult stem cells, but is not limited thereto.

The present invention provides a medium composition for differentiating from stem cells into NK cells, the medium composition including, as an effective ingredient, at least one selected from the group consisting of:
β-mercaptoethanol (BME, 2BME, 2-ME or β-met), sodium selenite, ethanolamine, ascorbic acid, IL-3, and adezmapimod (SB203580).

The present invention provides a kit for differentiating stem cells into NK cells, including the composition and instructions.

The present invention also provides a use of a composition for differentiating stem cells into NK cells or for promoting the differentiation of stem cells into NK cells, the composition including, as an active ingredient, one or more selected from the group consisting of:
β-mercaptoethanol (BME, 2BME, 2-ME, or β-met), sodium selenite, ethanolamine, ascorbic acid, IL-3, and adezmapimod (SB203580).

The present invention also provides a use of a composition for producing a preparation for differentiating stem cells into NK cells or for promoting the differentiation of stem cells into NK cells, the composition including, as an active ingredient, one or more selected from the group consisting of the following:
β-mercaptoethanol (BME, 2BME, 2-ME, or B-met), sodium selenite, ethanolamine, ascorbic acid, IL-3, and adezmapimod (SB203580).

The present invention also provides a pharmaceutical composition for preventing or treating cancer or an immune disease, including, as an active ingredient, NK cells expressing APC, differentiated by the method of the present invention.

The present invention also provides a method for preventing or treating cancer or an immune disease, or for enhancing the function of an anticancer agent or an immune disease therapeutic agent, the method including administering, to a subject in need thereof, NK cells expressing APC, which are differentiated by the method of the present invention, or a composition comprising the same as an active ingredient.

The present invention also provides a use of NK cells expressing APC, which are differentiated by the method of the present invention, or a composition including the same as an active ingredient, for preventing or treating cancer or an immune disease.

The present invention also provides a use of NK cells expressing APC, which are differentiated by the method of the present invention, or a composition including the same as an active ingredient, for preparing a drug for preventing or treating cancer or an immune disease.

### Advantageous Effects

It was confirmed that a culture method for increasing the differentiation efficiency and functionality of NK cells may increase NK cell differentiation efficiency by selecting and differentiating cells exhibiting specific characteristics. Additionally, the differentiation efficiency of NK cells was increased by changing the conditions of specific stages in the process of differentiating induced pluripotent stem cells into NK cells, the composition of stage-specific media used therein, and the treatment method of the composition. This method may be usefully applied not only as a means of quantitative increase, but also as a method representing qualitative improvement in that functionally enhanced NK cells may be obtained.

### BRIEF DESCRIPTION OF THE DRAWINGS

FIG. 1A is a schematic diagram of an experiment for confirming NK cell differentiation efficiency according to the expression of autofluorescent APC (Allophycocyanin) among hematopoietic progenitor cells (HPCs) differentiated from human induced pluripotent stem cells (iPSCs).

FIG. 1B shows the markers expressed by HPCs expressing autofluorescent APC on day 12 of HPC differentiation, and FIG. 1C shows the experimental result in which the cells were classified using a FACS sorter.

FIG. 1D shows the experimental result indicating the NK cell differentiation efficiency of total cells, APC-positive cells, and APC-negative cells after completion of the differentiation process.

FIG. 1E is an image showing the cytotoxicity assay result of total cells, APC-positive cells, and APC-negative cells after completion of the differentiation process, and FIG. 1F is a graph and table quantifying the result.

FIG. 2A is a schematic diagram of an experiment for confirming the effect of enhanced expression of activating receptors according to the change in the composition of the NK cell differentiation medium.

FIG. 2B shows the markers confirmed on days 10 to 14 of hematopoietic progenitor cell differentiation.

FIG. 2C shows the changes in the expression levels of various markers and activating receptors of NK cells obtained in Experiment 1 and Experiment 2 after completion of the NK cell differentiation process.

FIG. 2D is a graph and table showing the cytotoxic effect of functional NK cells obtained according to Experiment 2 of FIG. 2A.

FIG. 3A is a schematic diagram of an experiment for confirming the effect of decreased expression of inhibitory receptors according to the change in the composition of the NK cell differentiation medium.

FIG. 3B shows the markers confirmed on days 10 to 14 of hematopoietic progenitor cell differentiation.

FIG. 3C shows the changes in the expression levels of various markers and inhibitory receptors of NK cells obtained in Experiment 1 and Experiment 2 after completion of the NK cell differentiation process.

FIG. 3D is a graph and table quantitatively showing the changes in the expression levels of various markers confirmed in FIG. 3C.

FIG. 3E is a graph and table showing that the decrease in the expression level of NKG2A is statistically significant.

FIG. 3F is an image showing the cytotoxicity assay results of Experiment 1 and Experiment 2, and FIG. 3G is a graph and table quantifying the results.

FIG. 3H is an image showing the IFN-gamma ELISA analysis results of Experiment 1 and Experiment 2, and FIG. 31 is a graph and table quantifying the results.

### BEST MODES OF THE INVENTION

The present invention provides a method for differentiating NK cells from stem cells, the method including a step of selecting Allophycocyanin (APC)-expressing hematopoietic progenitor cells among hematopoietic progenitor cells cultured from stem cells. The method of the present invention may be a method for efficiently differentiating NK cells from stem cells.

In one embodiment of the present invention, the hematopoietic progenitor cells may additionally express one or more markers selected from the group consisting of CD34, CD31, CD43, and CD45, but are not limited thereto.

In the present invention, NK cells differentiated from cells expressing APC, which is autofluorescent, and expressing one or more markers selected from the group consisting of CD34, CD31, CD43, and CD45, which are markers of hematopoietic stem cells, exhibit significantly superior cytotoxic function, and thus may be usefully applied as an immunotherapeutic agent or a cell therapeutic agent.

In the present invention, “autofluorescent APC (allophycocyanin)” or “allophycocyanin (APC)” may mean a very bright phycobiliprotein isolated from red algae, which exhibits far-infrared fluorescence with a high quantum yield. It can be excited by laser lines of 594 and 633 nm and characterized by maximum absorbance at 650 nm and fluorescence emission peak at 660 nm, but is not limited thereto. It is generally known that the larger cells and the more granular cells, the more the number of fluorescent compounds increases, thus increasing autofluorescence, without being limited thereto.

In the present invention, “selection” may mean classifying hematopoietic progenitor cells that exhibit at least one of the characteristics of the APC or the markers from hematopoietic progenitor cells to differentiate NK cells from the hematopoietic progenitor cells, but is not limited thereto.

In one embodiment of the present invention, the differentiation efficiency of NK cells differentiated from the hematopoietic progenitor cells may be increased, but is not limited thereto.

In one embodiment of the present invention, the NK cells differentiated from the hematopoietic progenitor cells may have increased cytotoxic function, but are not limited thereto.

In the present invention, the method for differentiating NK cells from stem cells may include: 1) a step of culturing stem cells into hematopoietic progenitor cells; and 2) a step of culturing and differentiating the hematopoietic progenitor cells into NK cells, and the step of selecting hematopoietic progenitor cells expressing APC (Allophycocyanin) among the hematopoietic progenitor cells cultured from stem cells in the present invention may be additionally inserted between step 1) and step 2), and for example, may be expressed as follows, but is not limited thereto:

A method for differentiating NK cells from stem cells, comprising the following steps, wherein the method further comprises: 1-1) a step of selecting hematopoietic progenitor cells between step 1) and step 2):
1) a step of culturing hematopoietic progenitor cells cultured from stem cells; and
2) a step of differentiating NK cells from the hematopoietic progenitor cells.

Additionally, in the present invention, the method for differentiating NK cells from stem cells may include steps generally applied by those skilled in the art in the technical field of the present invention, for example, a step of pretreating stem cells, hematopoietic progenitor cells, and the like to be suitable for culturing, and a step of obtaining the differentiated NK cells.

The present invention provides a method for differentiating NK cells from stem cells, the method including: culturing hematopoietic progenitor cells and differentiating NK cells from the hematopoietic progenitor cells in a first NK cell differentiation medium or second NK cell differentiation medium including at least one selected from the group consisting of:
β-mercaptoethanol (BME, 2BME, 2-ME or B-met), sodium selenite, ethanolamine, ascorbic acid, and IL-3.

In one embodiment of the present invention, the second NK cell differentiation medium may not include IL-3, but is not limited thereto.

In one embodiment of the present invention, the first NK cell differentiation medium or the second NK cell differentiation medium may include, based on the total composition, 0.1 to 20 μM of β-mercaptoethanol, 0.1 to 50 ng/mL of sodium selenite, 1 to 600 μM of ethanolamine, and 1 to 200 mg/L of ascorbic acid, but is not limited thereto.

In the present invention, β-mercaptoethanol may be present in a concentration of 0.1 to 15 μM, 0.1 to 10 μM, 0.1 to 5 μM, 0.1 to 4 μM, 0.1 to 3 μM, 0.1 to 2 μM, 0.3 to 15 μM, 0.3 to 10 μM, 0.3 to 5 μM, 0.3 to 4 μM, 0.3 to 3 μM, 0.3 to 2 μM, 0.5 to 15 μM, 0.5 to 10 μM, 0.5 to 5 μM, 0.5 to 4 μM, 0.5 to 3 μM, or preferably 0.5 to 2 μM, but is not limited thereto.

In the present invention, sodium selenite may be present in a concentration of 0.1 to 25 ng/mL, 0.1 to 10 ng/ml, 0.1 to 8 ng/mL, 0.1 to 5 ng/mL, 0.5 to 50 ng/mL, 0.5 to 25 ng/ml, 0.5 to 10 ng/mL, 0.5 to 8 ng/mL, 0.5 to 5 ng/mL, 1 to 50 ng/mL, 1 to 25 ng/mL, 1 to 10 ng/ml, 1 to 8 ng/ml, or preferably 1 to 5 ng/ml, but is not limited thereto.

In the present invention, ethanolamine may be present in a concentration of 1 to 600 μM, 1 to 300 μM, 1 to 100 μM, 1 to 80 μM, 1 to 70 μM, 1 to 60 μM, 10 to 600 μM, 10 to 300 μM, 10 to 100 μM, 10 to 80 μM, 10 to 70 μM, 10 to 60 μM, 20 to 600 μM, 20 to 300 μM, 20 to 100 μM, 20 to 80 μM, 20 to 70 μM, 20 to 60 μM, 30 to 600 μM, 30 to 300 μM, 30 to 100 μM, 30 to 80 μM, 30 to 70 μM, or preferably 30 to 60 μM, but is not limited thereto.

In the present invention, ascorbic acid may be present in a concentration of 1 to 200 mg/L, 1 to 100 mg/L, 1 to 80 mg/L, 1 to 70 mg/L, 1 to 60 mg/L, 1 to 200 mg/L, 5 to 100 mg/L, 5 to 80 mg/L, 5 to 70 mg/L, 5 to 60 mg/L, 10 to 200 mg/L, 10 to 100 mg/L, 10 to 80 mg/L, 10 to 70 mg/L, or preferably 10 to 60 mg/L, but is not limited thereto.

In one embodiment of the present invention, the first NK cell differentiation medium or the second NK cell differentiation medium may further include one or more selected from the group consisting of stemline, stempro-34, SCF, IL-7, IL-15, and FLT3 ligand, but is not limited thereto.

In one embodiment of the present invention, the NK cells may have increased expression of an activating receptor, but are not limited thereto.

In one embodiment of the present invention, the activating receptor may be one or more selected from CD16 or NKG2D, but is not limited thereto.

In the present invention, CD16, which is an Fcγ receptor that recognizes the Fc portion of an IgG antibody specific for harmful cells, can function as an activating receptor that performs the function of inducing antibody-dependent cell-mediated cytotoxicity (ADCC).

In the present invention, NKG2D is a key stimulatory cell surface receptor, and as the activation stage for activating each immune cell is initiated when the NKG2D receptor binds to one of ligands, it can function as an activating receptor.

In one embodiment of the present invention, the second NK cell differentiation medium may further include a p38 MAPK inhibitor, but is not limited thereto.

In one embodiment of the present invention, the p38 MAPK inhibitor may be one or more selected from the group consisting of adezmapimod (SB203580), losmapimod, doramapimod, ralimetinib, and neflamapimod (VX-745), but is not limited thereto.

In the present invention, p38 MAPK may refer to a molecule known to play an essential role in the regulation of inflammatory signaling networks and in the biosynthesis of cytokines including tumor necrosis factor-α (TNF-α) and interleukin-1β (IL-1β), but is not limited thereto.

In one embodiment of the present invention, the NK cells may have reduced expression of an inhibitory receptors, but are not limited thereto.

In one embodiment of the present invention, the inhibitory receptor may be NKG2A, but is not limited thereto.

In one embodiment of the present invention, the NK cells may maintain or increase the expression level of one or more activating receptors selected from the group consisting of CD16, NKG2D, NKp30, and NKp44, but are not limited thereto.

In the present invention, NKG2, also known as CD159 (cluster of differentiation 159), is a receptor for natural killer (NK) cells. There are seven types of NKG2: A, B, C, D, E, F, and H. NKG2D is an activating receptor on the surface of NK cells, and NKG2A may refer to an inhibitory receptor that forms a heterodimer with CD94 to constitute the inhibitory receptor CD94/NKG2, but is not limited thereto.

In one embodiment of the present invention, the culturing performed in the first NK cell differentiation medium or the second NK cell differentiation medium may be performed from day 10 to 14 to day 52 to 56 of the entire incubation process, but is not limited thereto.

In one embodiment of the present invention, the culturing performed in the first NK cell differentiation medium may be performed from day 10 to 14 to day 16 to 20 of entire incubation process, but is not limited thereto.

In the present invention, the first NK cell differentiation medium was used in the first two administrations, and 15 mL was added each time, but is not limited thereto, and it may be used in an appropriate volume, concentration, and number of administrations within the above-described period.

In one embodiment of the present invention, the culturing performed in the second NK cell differentiation medium may be performed from day 16 to 20 to day 46 to 52 of entire incubation process, but is not limited thereto.

In the present invention, the second NK cell differentiation medium may not contain IL-3 or may additionally contain a p38 MAPK inhibitor, thereby increasing the differentiation efficiency of NK cells and enhancing the cytotoxic function of the differentiated NK cells.

In one embodiment of the present invention, the NK cells may exhibit increased cytotoxicity, but are not limited thereto.

In this specification, “stem cell” refers to a broad concept that collectively refers to undifferentiated cells that have the ability, i.e., stemness, to differentiate into various types of body tissue cells. These stem cells are primarily divided into embryonic stem cells that can be manufactured using embryos, mesenchymal stem cells, adult stem cells, induced pluripotent stem cells, and mesenchymal stem cells derived from induced pluripotent stem cells. Embryonic stem cells refer to a cell mass stage that occurs less than 14 days after fertilization before specific organs are formed. Recently, embryonic stem cells are also produced from normal cells through reverse differentiation. Therefore, they may be any cells that can differentiate into any cells or tissues that make up the body, without being limited thereto. Mesenchymal stem cells, which are extracted from umbilical cord blood, bone marrow, blood, etc., refer to primitive cells that are just before they differentiate into cells of specific organs such as bone, liver, and blood. Induced pluripotent stem cells are differentiated pluripotent stem cells that are produced by reverse differentiation from adult cells.

In one embodiment of the present invention, the stem cell may be one or more selected from the group consisting of induced pluripotent stem cells, embryonic stem cells, and adult stem cells, but is not limited thereto.

In the present invention, “proliferation (or expansion)” may mean an increase in the number of NK cells differentiated from stem cells or hematopoietic progenitor cells according to the NK cell differentiation method of the present invention, and the promotion/activation of the increase phenomenon. In addition, in the present invention, “differentiation” may mean a change from stem cells or hematopoietic progenitor cells to NK cells according to the NK cell differentiation method of the present invention.

The present invention provides a medium composition for differentiating from stem cells into NK cells, the medium composition including, as an effective ingredient, at least one selected from the group consisting of:
β-mercaptoethanol (BME, 2BME, 2-ME or β-met), sodium selenite, ethanolamine, ascorbic acid, IL-3, and adezmapimod (SB203580).

The present invention provides a kit for differentiating stem cells into NK cells, including the composition and instructions.

In the present invention, the instruction may describe the method of the present invention, but is not limited thereto.

In the present invention, the “kit” refers to a tool that enables the differentiation of NK cells by the method of the present invention. The kit of the present invention may include, in addition to the above substances, other components, compositions, solutions, or devices that are conventionally required for the storage and handling thereof. As a specific example, each component may be applied one or more times without limitation on the number of times, the order of applying each substance is not limited, and each substance may be applied simultaneously or sequentially.

In the present invention, the kit may include a container, instructions, and the like. The container may serve to package the above substance, and may also serve to store and secure it. The material of the container may take the form of, for example, a bottle, tub, sachet, envelope, tube, or ampoule, and may be formed partially or entirely from plastic, glass, paper, foil, wax, or the like. The container may be equipped with a cap that is initially a part of the container or is completely or partially detachable and attachable to the container by mechanical, adhesive, or other means, and may also be equipped with a stopper that allows access to the contents with a syringe needle. The kit may include an external package, and the external package may include instructions for use of the components.

In the present invention, when the term “comprising” is used, it does not exclude the presence of other components unless specifically stated otherwise, but rather means that other components may additionally be included. Throughout the present invention, the term “step of ˜ing” or “step of ˜” as used to describe the degree does not mean “a step for ˜.”

The present invention also provides a pharmaceutical composition for preventing or treating cancer or an immune disease, including, as an active ingredient, NK cells expressing APC, differentiated by the method of the present invention.

The pharmaceutical composition for preventing or treating cancer or an immune disease according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO3) carbon dioxide gas, sodium metabisulfite (Na2S2O5), sodium sulfite (Na2SO3), nitrogen gas (N2), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter +cholesterol, lecithin, lanette wax, glycerol monostearate, Tween or span, imhausen, monolan (propylene glycol monostearate), glycerin, Adeps solidus, buytyrum Tego-G, cebes Pharma 16, hexalide base 95, cotomar, Hydrokote SP, S-70-XXA, S-70-XX75 (S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases.

As used herein, the “subject” refers to a subject in need of treatment of a disease, and more specifically, refers to a mammal such as a human or a non-human primate, a mouse, a rat, a dog, a cat, a horse, and a cow.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “alleviation” as used herein means all actions that reduce the degree of parameters related to a target disease, e.g., symptoms via administration of the composition according to the present invention.

In the present invention, NK cells can exhibit therapeutic effects even in a single form, and when they are administered simultaneously or sequentially with other drugs (for example, when the disease is cancer, a chemotherapy agent, a targeted anticancer agent, an immune anticancer agent, or a metabolic anticancer agent), the anticancer effect can be enhanced.

Therefore, the present invention can provide an anticancer auxiliary pharmaceutical composition containing NK cells as an effective ingredient. Here, the term “anticancer auxiliary pharmaceutical composition” may mean, but is not limited to, a composition that causes or enhances an anticancer effect when administered in combination with an anticancer drug.

In addition, the “anticancer effect” may mean the effect of the anticancer agent itself, i.e., causing or enhancing the responsiveness to the anticancer agent, and may mean causing or enhancing the effect of combination treatment with other anticancer agents or anticancer treatment methods, but is not limited thereto.

The anticancer auxiliary pharmaceutical composition of the present invention can be administered in combination with an anticancer agent, and the timing, cycle, dosage, concentration, method, etc. of the combined use of the anticancer auxiliary pharmaceutical composition of the present invention can be applied in the most preferable manner by considering the clinical condition of a patient to whom the composition is administered, the type of cancer, the type of anticancer agent to be combinedly administered, the type of other compositions, etc.

In addition, the anticancer auxiliary pharmaceutical composition can be prepared together with other substances that can be generally added to enhance the auxiliary effect of anticancer drugs, and can be administered in combination with other compositions.

In addition, pretreatment or post-treatment methods, storage methods, and usage methods that are generally performed to enhance auxiliary effects can be applied without limitation.

The present invention may also provide a pharmaceutical composition for combination administration with an anticancer agent, comprising NK cells as an active ingredient.

### Modes of the Invention

Hereinafter, preferable examples are presented to aid in the understanding of the present invention. However, these examples are provided merely for easier understanding of the present invention, and the content of the present invention is not limited by these examples.

### EXAMPLE

### Example 1. Improvement of iNK Differentiation Efficiency Through Autofluorescence-Based Sorting During Differentiation

### Example 1-1. Culture of Induced Pluripotent Stem Cells

### 1.1. Culture Vessel Coating

iMatrix-511 (175 μg) was taken out and added in an amount of 9.6 ul to 1.5 ml of DPBS in each well of 6 wells, allowing it to be coated for 1 hour in a coating incubator. Subculture was then performed.

### 1.2. Preparation of Culture Medium (Stemfit004)

The culture medium was thawed at room temperature (never warmed to 37° C.), and 1 ml of the antibiotic Primocin was added to and mixed with stemfit004 to prepare a complete medium.

1.3. Preparation of 10 mM y-27632

y-27632 was prepared in DW to make 10 mM, then aliquoted into EP tubes and stored at −20° C.

### 1.4. Method of Culturing Induced Pluripotent Stem Cells

The complete medium was prepared by warming at room temperature for 30 minutes or more.

### 1.4.1 General Culture

The medium of cells cultured in a 6-well plate was removed, and the culture medium was replaced by 2.5 ml every day.

### 1.4.2 Subculture

10 mMy-27632 was dissolved at room temperature, and then added at a concentration of 1000x to the complete medium warmed at room temperature. The culture medium in the plate containing the cells to be subcultured was removed.

1 mL of DPBS was added to each well of a 6-well plate and washed. 1 mL of TrypLE reagent was added and incubated for 15 minutes (37° C., 5% CO₂). After confirming that the cells were detached, 1 ml of the reacted TrypLE reagent was added to a 15 ml tube containing 8 ml of the complete media, and the media was added once more to collect the cells and put them into the 15 ml tube again. Next, the tube was centrifuged at 300 g for 5 minutes, and the media was removed, leaving only the cells. Next, the media+y-26532 (3 ml) was added and the cells were counted. 3×10\hat{ }4 cells/well/2.5 ml were seeded for 6 wells.

### Example 1-2. Differentiation into NK Cells from Induced Pluripotent Stem Cells

### 2.1. Culture Vessel Coating

iMatrix-511 (175 μg) was taken out and added in an amount of 75 μl to 15 ml of DPBS in a T-75 flask, allowing it to be coated for 1 hour in a coating incubator.

### 2.2. Stemfit004

The culture medium was thawed at room temperature (never warmed to 37° C.), and 1 ml of the antibiotic Primocin was added to and mixed with stemfit004 to prepare a proliferation complete medium.

2.3. Preparation of 10 mM y-27632

y-27632 was prepared in DW to make 10 mM, then aliquoted into EP tubes and stored at −20° C.

2.4. Differentiation into NK Cells from Induced Pluripotent Stem Cells

The proliferation complete medium was prepared by warming it at room temperature for 30 minutes or more.

### 2.4.1 Spheroid Production

Spheroids were produced using a SPHERICAL PLATE 5D plate. 7.5×10\hat{ }5 cells were seeded per well. The seeding method was applied in the same manner as in 1.4.2. Next, incubation was performed for 24 hours at 37° C., 5% CO₂.

### 2.4.2 Spheroid Seeding

The produced spheroids were seeded in the coated T-75 flasks. A total of 15 ml of media was seeded per well of 7 T-75 flasks. Next, the culture was performed until the colony size reached 750 μm to 100 μm (normal culture for 3 to 5 days).

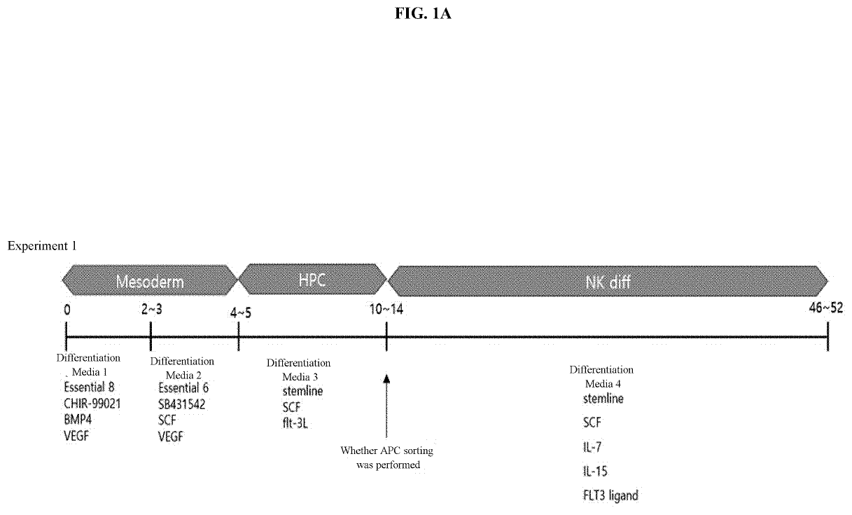

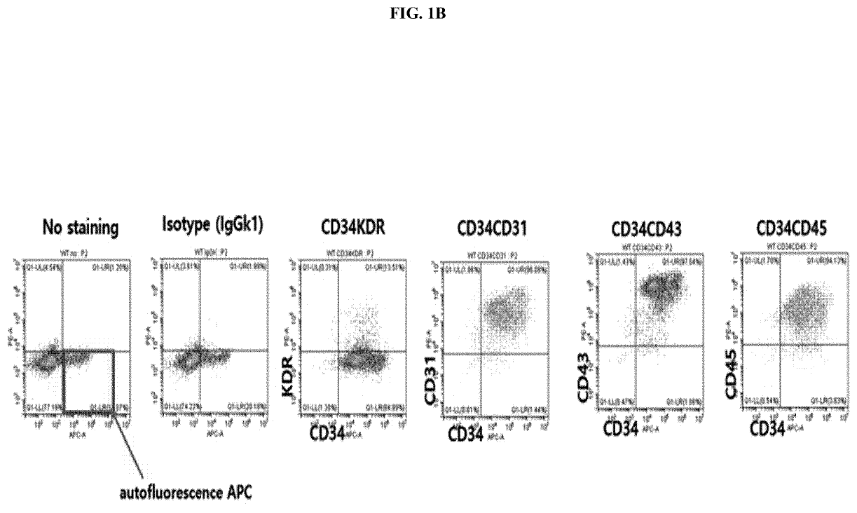

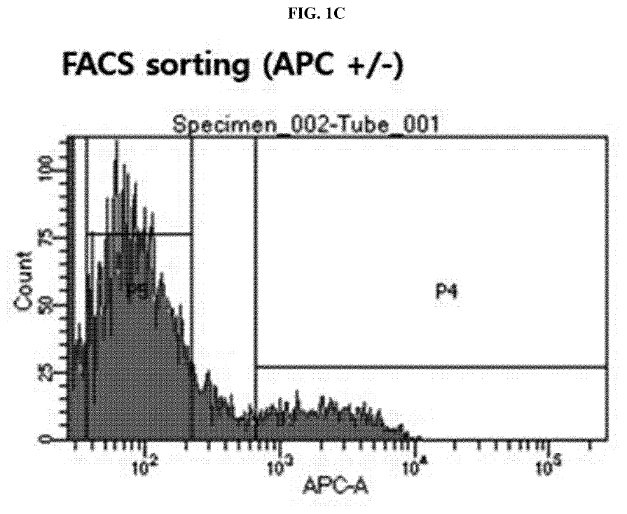

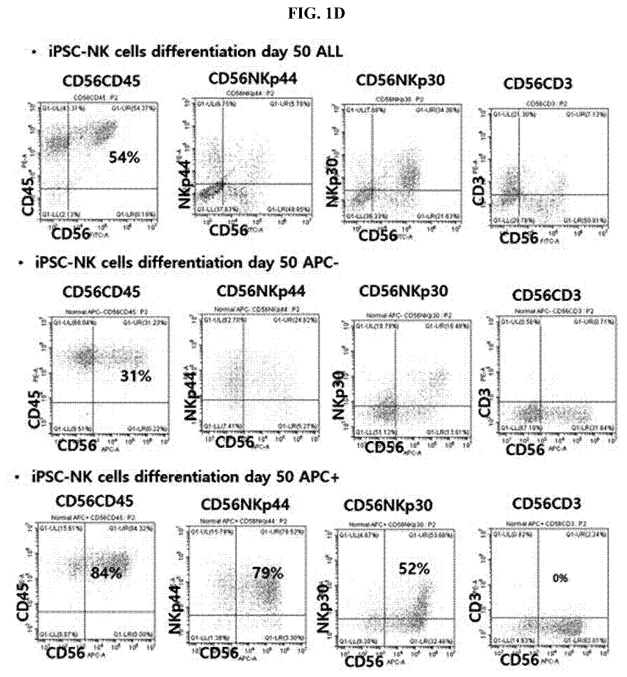

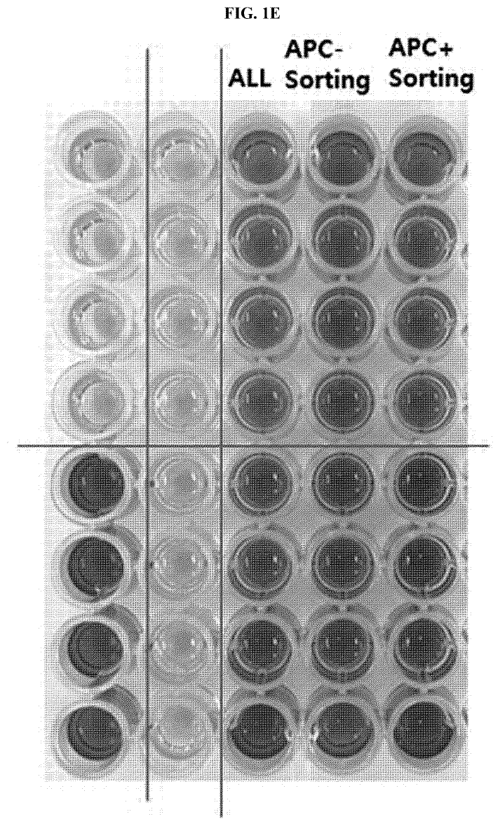

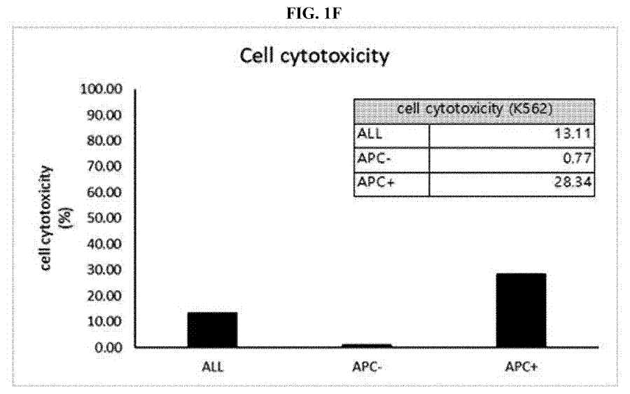

2.4.3 Schematic Diagram of Differentiation into NK Cells (Scheme, FIG. 1A)
2.4.3.1. Differentiation process into NK Cells
2.4.3.1.1. 0 to 52 day differentiation process

Day 0: Remove growth media (Stemfit 004) by suction, wash with Essential 8 media, add 15 ml of Differentiation Media 1, and incubate.

Day 2 to 3: Remove Differentiation Media 1 by suction, add 15 ml of Differentiation Media 2, and incubate.

Day 4 to 5: Remove Differentiation Media 2 by suction, add 15 ml of Differentiation Media 3, and incubate.

Day 8 to 9: Add 15 ml of Differentiation Media 3 (total vol 30 ml).

Day 10 to 14: Harvest media in T75 during differentiation (30 ml/T75).
Group 1: After centrifugation at 300 g for 5 min, add 15 ml of Differentiation Media 4 (15 ml/T75).
Groups 2 and 3: Classify the harvested cells into cells expressing APC and cells not expressing APC using the FACS sorting function. Next, add 15 ml of Differentiation Media 4 to the original flask for seeding.

Day 12 to 16: Add 15 ml of Differentiation Media 4 (total vol 30 ml)

Day 18 to 48: Next, change half of the media once every 4 days. Take 15 ml per T75, put it in a 50 ml tube, and centrifuge it at 300 g for 5 minutes. Next, remove the media, add 15 ml of new Differentiation Media 4 to release cells, and add it to a flask.

Day 46 to 52: Differentiation completion. Cell harvest for analysis. The media in the flask was harvested and placed in a 50 ml tube, and then centrifuged at 300 g for 5 minutes. Next, the differentiation media was added to conduct cell counting (cell counter X, manual counting), and the number of cells for each analysis was confirmed.

TABLE 1

| Analysis | Number | | | |
|---|---|---|---|---|
| Day | of tubes | Marker | Fluorescence | Remarks |
| day 0 | tube 1 | no | | |
| | tube 2 | IgGM | PE | isotype |
| | tube 3 | TRA-1-81 | PE | iPSC marker |
| | tube 4 | TRA-1-60 | PE | iPSC marker |
| | tube 5 | IgG3 | PE | isotype |
| | tube 6 | SSEA4 | PE | iPSC marker |
| day 10~14 | tube 1 | no | | compensation |
| | tube 2 | CD31 | PE | compensation |
| | tube 3 | CD34 | APC | compensation |
| | tube 4 | IgGK1 | APC/PE | isotype |
| | tube 5 | CD34KDR | APC/PE | HPC marker |
| | tube 6 | CD34CD31 | APC/PE | HPC marker |
| | tube 7 | CD34CD43 | APC/PE | HPC marker |
| | tube 8 | CD34CD45 | APC/PE | HPC marker |
| | tube 9 | CD56CD45 | APC/PE | NK marker |
| day 34~38 | tube 1 | no | | compensation |
| | tube 2 | CD45 | PE | compensation |
| | tube 3 | CD56 | APC | compensation |
| | tube 4 | IgGK1 | APC/PE | isotype |
| | tube 5 | CD34CD31 | APC/PE | HPC marker |
| | tube 6 | CD34CD43 | APC/PE | HPC marker |
| | tube 7 | CD34CD45 | APC/PE | HPC marker |
| | tube 8 | CD56CD45 | APC/PE | NK marker |
| | tube 9 | CD56NKG2A | APC/PE | NK marker |
| | tube 10 | CD56NKG2D | APC/PE | NK marker |
| | tube 11 | CD56NKp30 | APC/PE | NK marker |
| | tube 12 | CD56NKp44 | APC/PE | NK marker |
| | tube 13 | CD56NKp46 | APC/PE | NK marker |
| | tube 14 | CD56CD7 | APC/PE | NK marker |
| | tube 15 | CD56CD16 | APC/PE | NK marker |
| | tube 16 | CD56CD3 | APC/PE | NK marker |
| day 46~52 | tube 1 | no | | compensation |
| | tube 2 | CD45 | PE | compensation |
| | tube 3 | CD56 | APC | compensation |
| | tube 4 | IgGK1 | APC/PE | isotype |
| | tube 5 | CD56CD45 | APC/PE | NK marker |
| | tube 6 | CD56NKG2A | APC/PE | NK marker |
| | tube 7 | CD56NKG2D | APC/PE | NK marker |
| | tube 8 | CD56NKp30 | APC/PE | NK marker |
| | tube 9 | CD56NKp44 | APC/PE | NK marker |
| | tube 10 | CD56NKp46 | APC/PE | NK marker |
| | tube 11 | CD56CD7 | APC/PE | NK marker |
| | tube 12 | CD56CD16 | APC/PE | NK marker |
| | tube 13 | CD56CD3 | APC/PE | NK marker |
| | tube 14 | CD56KIR(a, h, g) | APC/PE | NK marker |
| | tube 15 | CD56KIR(b1, b2) | APC/PE | NK marker |
| | tube 16 | CD56KIR(e1, e2) | APC/PE | NK marker |
| | tube 17 | IgGM | PE | isotype |
| | tube 18 | TRA-1-81 | PE | iPSC marker |
| | tube 19 | TRA-1-60 | PE | iPSC marker |
| | tube 20 | IgG3 | PE | isotype |
| | tube 21 | SSEA4 | PE | iPSC marker |

2.4.3.2 FACS verification process of differentiation from stem cells into NK cells (Table 1)
2.4.3.2.1 At each stage, cells undergoing differentiation were harvested and centrifuged at 300 g for 5 minutes.
2.4.3.2.2 Next, after removing the media, 10 ml of FACS buffer was added and washed by centrifugation at 300 g for 5 minutes.
2.4.3.2.3 1x10\hat{ }5 cells were added to the tube.
2.4.3.2.4 1 μl of each antibody and isotype was added to the tube.
2.4.3.2.5 Incubation was conducted on ice for 1 hour.
2.4.3.2.6 Next, 1 ml of FACS buffer was added, and centrifugation was conducted at 300 g for 5 minutes to wash.
2.4.3.2.7 The method of 2.4.3.2.6 was repeated three times.
2.4.3.2.8 Next, measurement was conducted with FASC device.
2.4.3.3 Verification process of differentiation from stem cells to NK cells (cell cytotoxicity) (FIG. 1B)
2.4.3.3.1 Target cell number: K562 1×10\hat{ }4/well/Effector cell: iNK cells 1×10\hat{ }5 cells/well, E: T ratio=10:1
2.4.3.3.2 Media: stempro-34+SCF+FLT3-L+IL7+IL15
2.4.3.3.3 Cells were seeded in a U-bottom 96-well plate according to the cytotoxicity scheme in FIG. 1B.
2.4.3.3.4 Next, incubation was performed for 3 hours and 15 minutes.
2.4.3.3.5 The incubated plate was taken out, and a lysis buffer was added in 10 μl increments (maintaining a constant pipetting rate).
2.4.3.3.6 Next, incubation was performed for 45 minutes.
2.4.3.3.7 After incubation, centrifugation was performed at 300 g for 5 minutes.
2.4.3.3.8 After centrifugation, 50 μl was transferred to a new flat 96-well plate.
2.4.3.3.9 50 μl of pre-dissolved substrate solution was added.
2.4.3.3.10 Next, incubation was performed for 30 minutes.
2.4.3.3.11 After incubation, 50 μl of stop solution was added, and measurement was conducted at 490 nm using a microplate reader (air bubbles were removed because the measurement value would bounce if there were air bubbles in the well).

### Example 1-3. Confirmation of Increased NK Cell Differentiation Efficiency Depending on Presence or Absence of Autofluorescent APC Expression

Autofluorescence APC was identified during iPSC-HPC differentiation at diff day 10-14 of hematopoietic progenitor cells (HPC) differentiation of human induced pluripotent stem cells (FIG. 1B). FACS sorting was performed while classifying APC expression (+) and APC non-expression (−) according to expression status. Additionally, they were classified into APC +(P4) and APC−(P5) using a FACS sorter (FIG. 1C).

As a result, it was confirmed that the differentiation efficiency from APC-expressing HPCs to NK cells was superior to that from APC-nonexpressing HPCs to NK cells (FIG. 1D).

In addition, the cell cytotoxicity of APC-expressing NK cells (APC+) and APC-nonexpressing NK cells (APC-) among NK cells that had been differentiated was analyzed.

As a result, it was confirmed that NK cells differentiated from APC-expressing HPCs had high cytotoxicity (FIGS. 1E and 1F).

### Example 2. Confirmation of Effect of Increasing Expression of Activating Receptors Dependent Upon Change in Composition of NK Cell Differentiation Medium

### Example 2-1. Culture of Human Induced Pluripotent Stem Cells

iPSC were cultured in the same manner as in Example 1-1 of Example 1.

### Example 2-2. Differentiation from Human Induced Pluripotent Stem Cells into NK Cells

Differentiation was conducted in the same manner as in the methods 2.1 to 2.4.2 of Example 1-2 of Example 1.

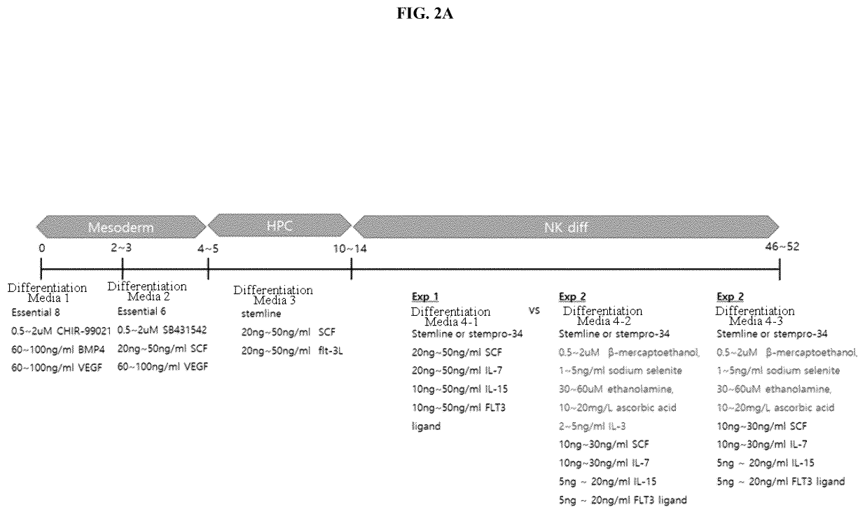

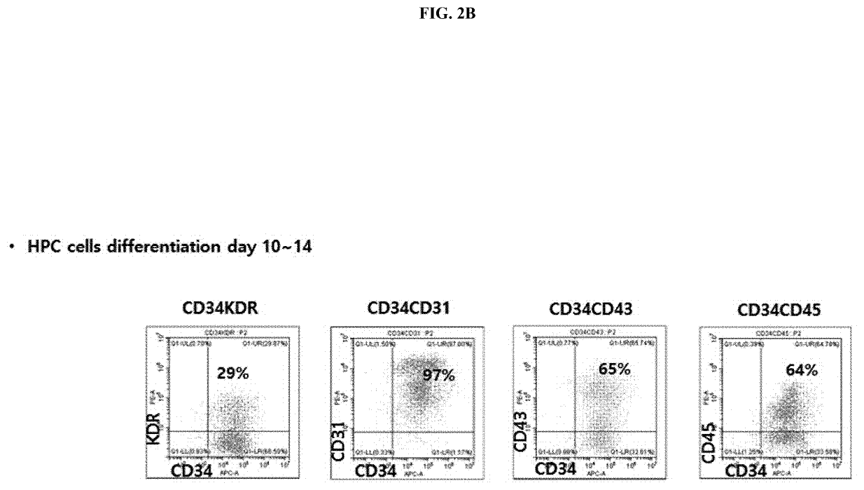

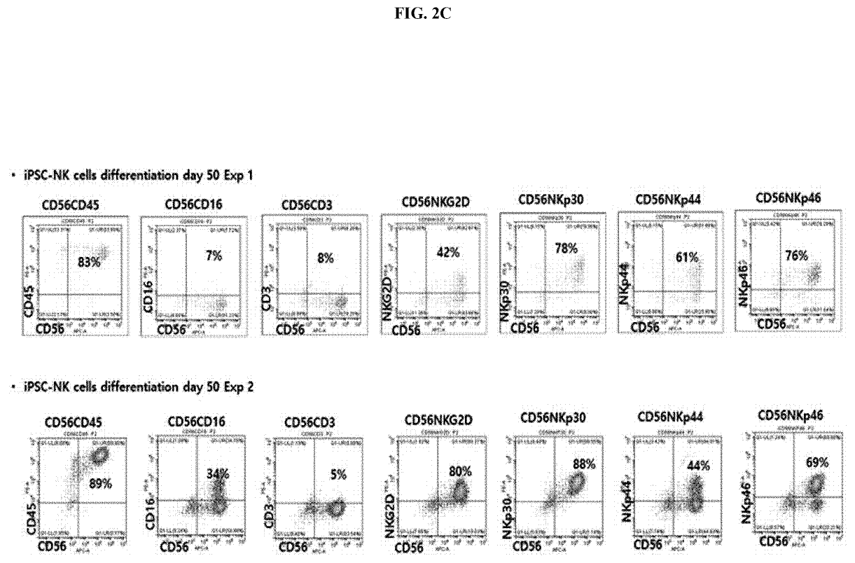

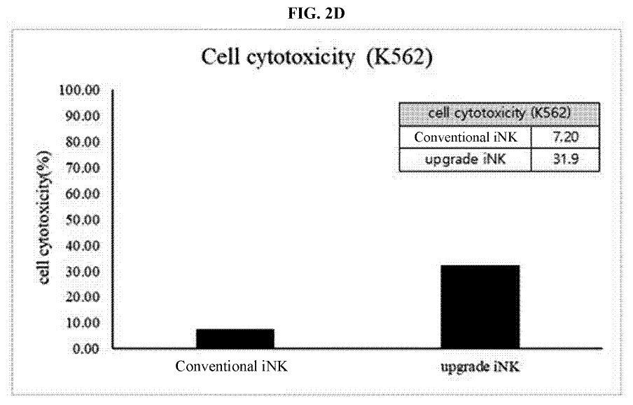

2.4.3 Schematic Diagram of Differentiation into NK Cells (FIG. 2A)
2.4.3.1 differentiation process into NK Cells
2.4.3.1.1 Day 0: Remove growth media (Stemfit 004) by suction, wash with Essential 8 media, add 1 vol 15 ml of differentiation media, and incubate.

Day 2 to 3: Remove Differentiation Media 1 by suction, add 15 ml of Differentiation Media 2, and incubate.

Day 4 to 5: Remove Differentiation Media 2 by suction, add 15 ml of Differentiation Media 3, and incubate.

Day 8 to 9: Add 15 ml of Differentiation Media 3 (total vol 30 ml).

Day 10 to 14: Harvest media in T75 during differentiation (30 ml/T75).

### Exp 1

: After centrifugation at 300 g for 5 min, add 15 ml of Differentiation Media 4-1 (15 ml/T75).

: Day 12 to 16: Add 15 ml of Differentiation Media 4-1 (15 ml/T75).

: Day 16 to 46: Next, change half of the media once every 4 days. Put 15 ml of each T75 into a 50 ml tube and centrifuge at 300 g for 5 minutes. Next, remove the media, add 15 ml of new Differentiation Media 4-1 to release the cells, and add it to a flask.

### Exp 2

: After centrifugation at 300 g for 5 min, add 15 ml of Differentiation Media 4-2 (15 ml/T75).

: Day 12 to 16: Add 15 ml of Differentiation Media 4-2 (total vol 30 ml).

: Day 16 to 20: Completely change the media with Differentiation Media 4-3. After centrifugation at 300 g for 5 min, add 15 ml of Differentiation Media 4-3 (excluding IL-3).

Day 18 to 22: Add 15 ml of Differentiation Media 4-3.

Day 26 to 48: Next, change half of the media once every 4 days. Put 15 ml of each T75 into a 50 ml tube and centrifuge at 300 g for 5 minutes. Next, remove the media, add 15 ml of new Differentiation Media 4-3 to release cells, and add it to a flask.

Common Day 46 to 52: Differentiation terminated. Harvest cells for various analyses. Harvest the media in the flask and add it to a 50 ml tube. Next, centrifuge at 300 g for 5 minutes, add a differentiation media, count cells (cell counter not used, proceed with manual counting), and check the number of cells for each analysis.

A differentiation verification process was performed in the same manner as in the methods 2.4.3.2 and 2.4.3.3 of Example 1-2 of Example 1.

### Example 2-3. Confirmation of Effect of Increasing Expression of Activating Receptors Dependent Upon Change in Composition of NK Cell Differentiation Medium

The expression of each marker on days 10 to 14 of hematopoietic progenitor cell differentiation is shown in FIG. 2B. As shown in the drawing, it was confirmed that HPC differentiation proceeded without any problems at the HPC stage.

The effect of increasing the expression of activating receptors, dependent upon the composition change of Differentiation Media 4-1, 4-2, and 4-3, of NK cells using differentiated HPC was analyzed. Here, CD16 and NKG2D were analyzed as activating receptors. In addition, the change in the expression level of each marker dependent upon the medium compositions of Experiment 1 (Exp 1) and Experiment 2 (Exp 2) after the NK cell differentiation process was completed was analyzed.

As a result, the change in the expression level of each marker was confirmed as shown in FIG. 2C. As shown in the drawing, it was confirmed that the expression of CD16 and NKG2D significantly increased by about 5 times and 2 times, respectively.

In addition, the cytotoxicity analysis results showed that the cytotoxicity of functional NK cells with significantly increased CD16 and NKG2D expression increased approximately 4.5-fold (FIG. 2D).

Therefore, it was confirmed that functionally superior NK cells can be obtained by changing the composition of the NK cell differentiation medium composition applied to Stage 3 of the present invention and the method of using the medium.

### Example 3. Confirmation of Effect of Reducing Expression of Inhibitory Receptors Dependent Upon Change in Composition of NK Cell Differentiation Medium

### Example 3-1. Culture of Human Induced Pluripotent Stem Cells

iPSC were cultured in the same manner as in Example 1-1 of Example 1.

### Example 3-2. Differentiation from Human Induced Pluripotent Stem Cells into NK Cells

Differentiation was conducted in the same manner as in the methods 2.1 to 2.4.2 of

### Example 1-2 of Example 1

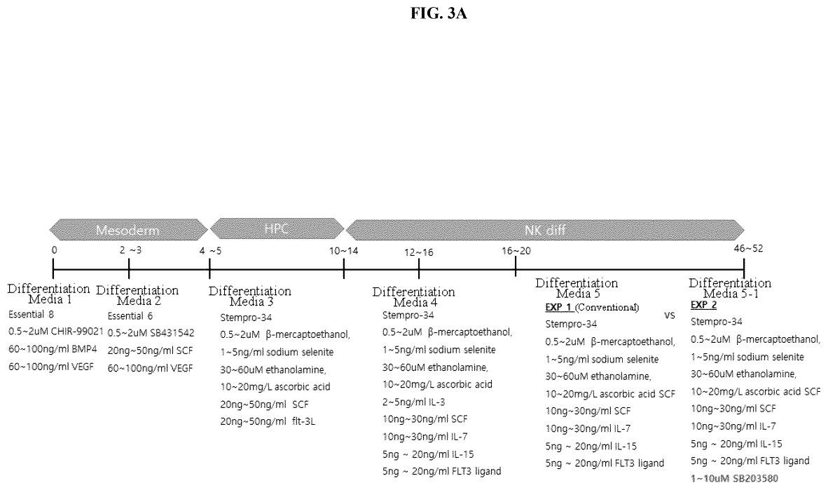

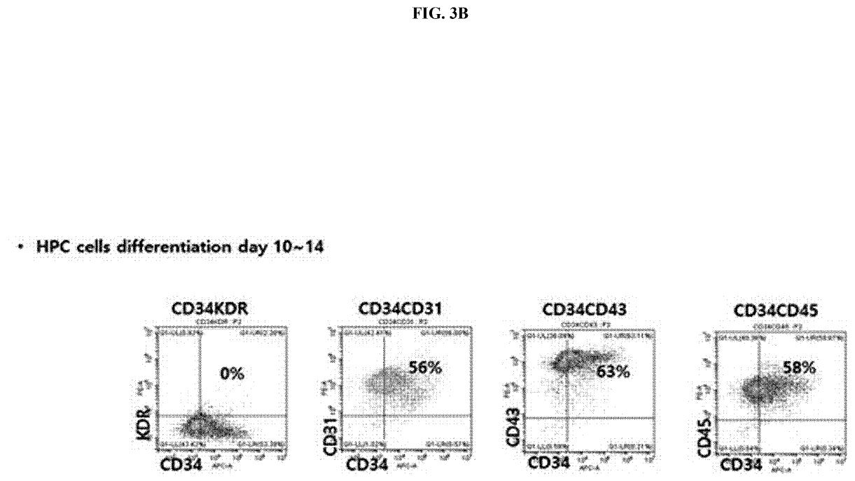

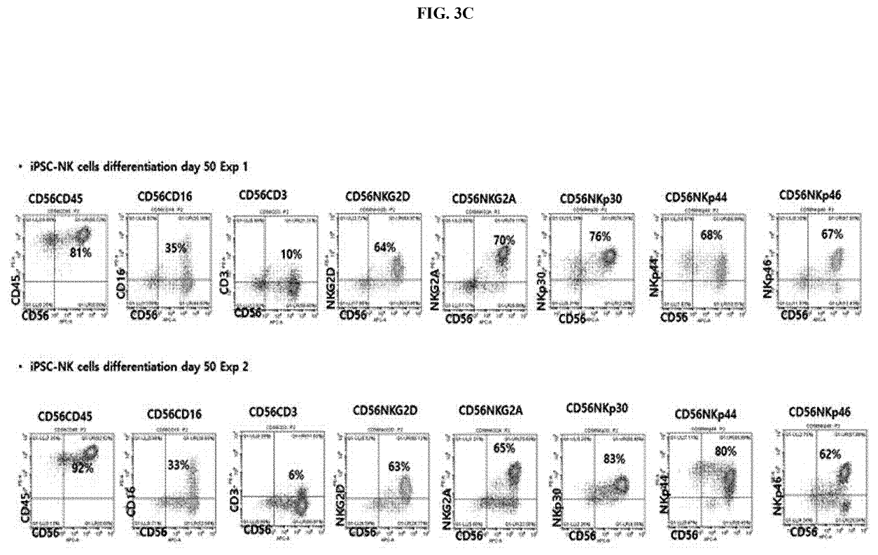

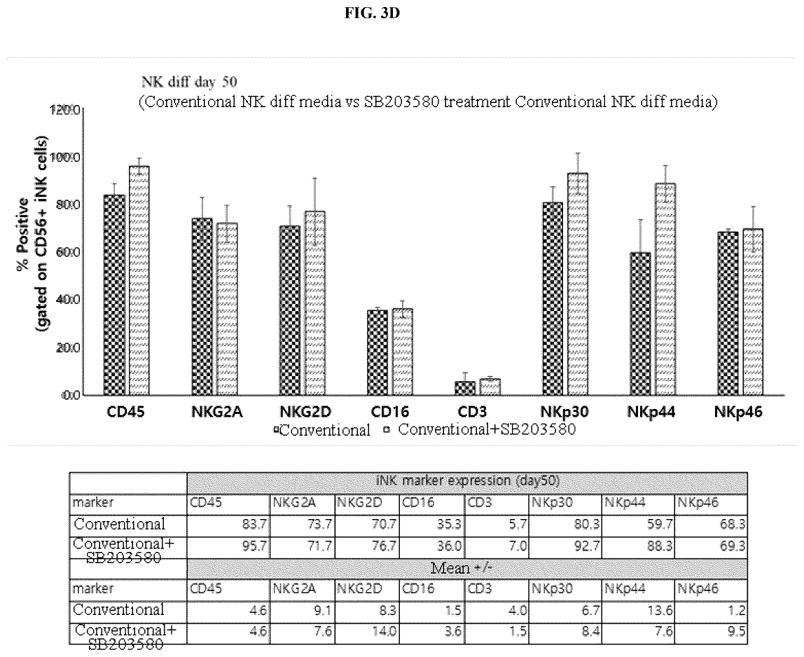

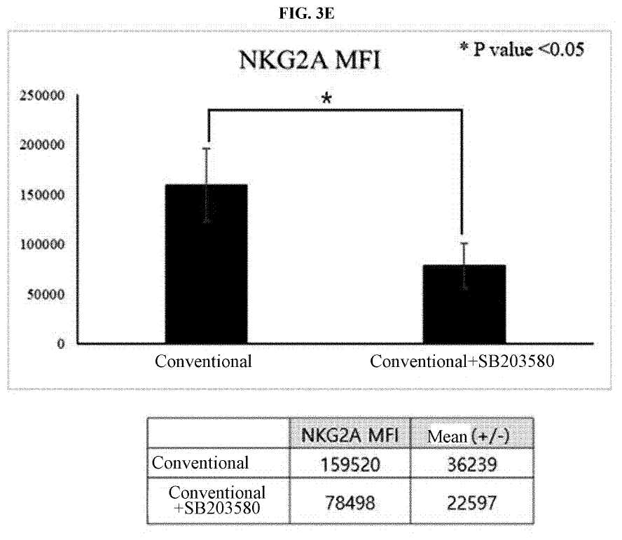

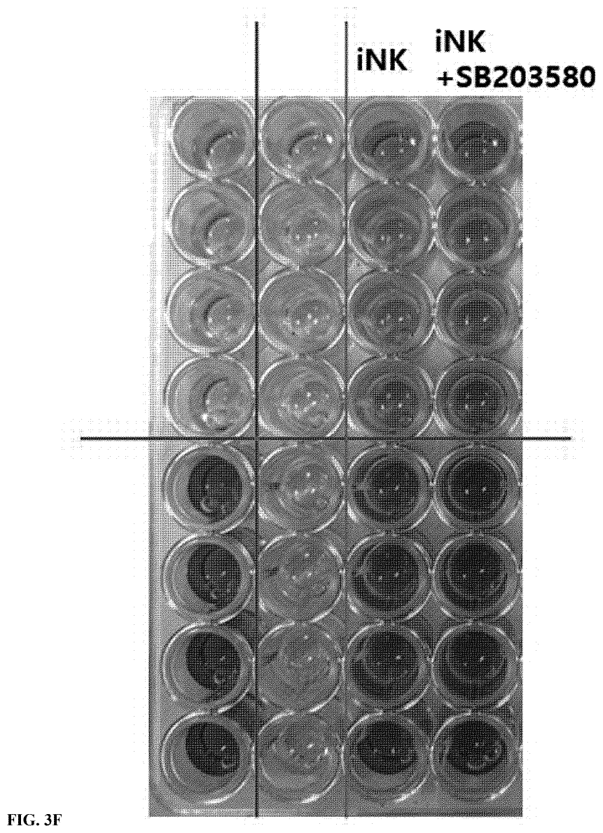

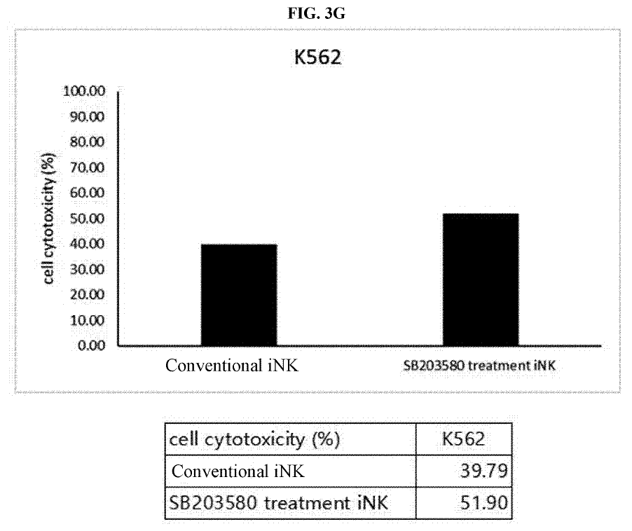

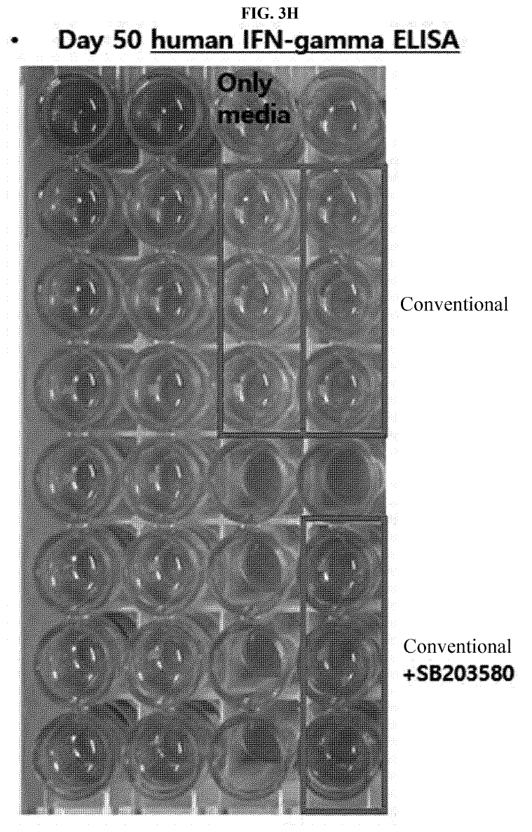

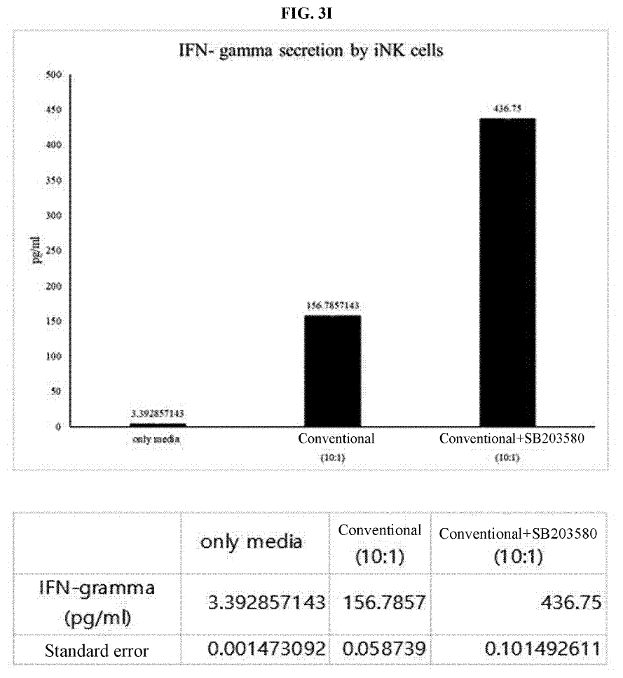

2.4.3 Schematic Diagram of Differentiation into NK Cells (FIG. 3A)
2.4.3.1 Differentiation process into NK cells
2.4.3.1.1 Day 0: Remove growth media (Stemfit 004) by suction, wash with Essential 8 media, add 1 vol 15 ml of differentiation media, and incubate.

Day 2 to 3: Remove Differentiation Media 1 by suction, add 15 ml of Differentiation Media 2, and incubate.

Day 4 to 5: Remove Differentiation Media 2 by suction, add 15 ml of Differentiation Media 3, and incubate.

Day 6 to 7: Add 15 ml of Differentiation Media 3-1 (total vol 30 ml).

Day 8 to 10: Change half of the media. Harvest 15 ml of the media in the flask and add it to a 50 ml tube. Next, centrifuge at 300 g for 5 minutes, remove the media, and add Differentiation Media 3-1 to the flask during differentiation. Next, incubate.

Day 10 to 14: Harvest the media in T75 during differentiation (30 ml/T75), centrifuge at 300 g for 5 min, and add 15 ml of Differentiation Media 4 (15 ml/T75).
2.4.3.1.1 Day 12 to 16: Add 15 ml of Differentiation Media 4 (total vol 30 ml).

### Exp 1

Day 16 to 20: Completely change from Differentiation Media 4 to Differentiation Media 5. Centrifuge at 300 g for 5 min, and then add 15 ml of Differentiation Media 5.

Day 18 to 22: Add 15 ml of Differentiation Media 5.

Day 26 to 48: Next, change half of the media once every 4 days. Put 15 ml of each T75 into a 50 ml tube and centrifuge at 300 g for 5 minutes. Next, remove the media, add 15 ml of new Differentiation Media 5 to release cells, and add it to a flask.

Day 46 to 52: Differentiation terminated. Harvest cells for analysis. Harvest the media in the flask and add it to a 50 ml tube. Next, centrifuge at 300 g for 5 minutes, add a differentiation media, count cells (cell counter not used, proceed with manual counting), and check the number of cells for each analysis.

### Exp 2

Day 16 to 20: Completely change from Differentiation Media 4 into Differentiation Media 5-1. After centrifugation at 300 g for 5 min, add 15 ml of Differentiation Media 5-1.

Day 18 to 22: Add 15 ml of Differentiation Media 5-1.

Day 26 to 48: Next, change half of the media once every 4 days. Put 15 ml of each T75 into a 50 ml tube and centrifuge at 300 g for 5 minutes. Next, remove the media, add 15 ml of new Differentiation Media 5-1 to release cells, and add it to a flask.

Day 46 to 52: Differentiation terminated. Harvest cells for analysis. Harvest the media in the flask and add it to a 50 ml tube. Next, centrifuge at 300 g for 5 minutes, add a differentiation media, count cells (cell counter not used, proceed with manual counting), and check the number of cells for each analysis.

A differentiation verification process was performed in the same manner as in the methods 2.4.3.2 and 2.4.3.3 of Example 1-2 of Example 1.
2.4.3.4 Verification process of differentiation from stem cells to NK cells (IFN-gramma EILSA)
2.4.3.4.1 Target cell number: K562 5×10\hat{ }4/well, Effector cell: iNK cells 1×10\hat{ }6 cells/well, E: T ratio=20:1
2.4.3.4.2 Media: stempro-34+SCF+FLT3-L+IL7+IL15
2.4.3.4.3 After dispensing K562 (target cells) and iNK cells (effector cells) into 24 wells, incubate for 4 hours.
2.4.3.4.4 After the reaction is terminated, harvest the media.
2.4.3.4.5 Centrifuge the harvested media at 300 g for 5 minutes, and replace the media, leaving only the pellet in a new EP tube.
2.4.3.4.6 Next, measure IFN-gamma using Human IFN-gamma Quantikine ELISA Kit (R&D, Cat. DIF50C) according to the kit protocol.

### Example 3-3. Confirmation of Effect of Reducing Expression of Inhibitory Receptors Dependent Upon Change in Composition of NK Cell Differentiation Medium

As a result, the expression of each marker on days 10 to 14 of hematopoietic progenitor cell differentiation was as shown in FIG. 3B. Similar to the experimental results of Example 2-3, it was confirmed that HPC differentiation proceeded without any problems at the HPC stage.

In addition, the change in the level of each marker dependent upon the medium compositions of Experiment 1 (Exp 1) and Experiment 2 (Exp 2) after the NK cell differentiation process was completed was confirmed as shown in FIG. 3C. In addition, the quantification results thereof are shown in FIG. 3. As shown in the drawing, the level of NKG2A was slightly reduced from 70% to 65%, but, when the MFI value for NKG2A was checked, it was confirmed that the reduction level of NKG2A was statistically significant when the medium composition was changed according to Example 3 (FIG. 3E).

In addition, the cytotoxicity analysis results showed that the cytotoxicity of the obtained NK cells increased when the medium composition was changed according to this example (FIGS. 3F and 3G). In addition, the IFN-gamma ELISA analysis results confirmed that the functionality of the obtained NK cells was significantly increased (FIGS. 3H and 31).

Therefore, it was confirmed that functionally superior NK cells can be obtained by changing the composition of the NK cell differentiation medium composition applied to Stage 3 of the present invention.

The foregoing description of the present invention is provided by way of example, and those skilled in the art to which the present invention pertains will understand that various modifications can be easily made in other specific forms without departing from the technical spirit or essential characteristics of the present invention. Therefore, the embodiments described above should be understood as illustrative in all respects and not as limiting.

### INDUSTRIAL APPLICABILITY

It was confirmed that a culture method for increasing the differentiation efficiency and functionality of NK cells may increase NK cell differentiation efficiency by selecting and differentiating cells exhibiting specific characteristics. Additionally, the differentiation efficiency of NK cells was increased by changing the conditions of specific stages in the process of differentiating induced pluripotent stem cells into NK cells, the composition of stage-specific media used therein, and the treatment method of the composition. This method may be usefully applied not only as a means of quantitative increase, but also as a method representing qualitative improvement in that functionally enhanced NK cells may be obtained, and thus has industrial applicability.

1. A method of differentiating NK cells from stem cells, the method comprising selecting allophycocyanin (APC)-expressing hematopoietic progenitor cells from among hematopoietic progenitor cells cultured from stem cells.

2. The method according to claim 1, wherein the hematopoietic progenitor cells additionally express one or more markers selected from the group consisting of CD34, CD31, CD43, and CD45.

3. The method according to claim 1, wherein differentiation efficiency of NK cells differentiated from hematopoietic progenitor cells increases.

4. The method according to claim 1, wherein NK cells differentiated from hematopoietic progenitor cells have increased cytotoxic function.

5. A method of differentiating NK cells from stem cells, the method comprising: culturing hematopoietic progenitor cells and differentiating NK cells from the hematopoietic progenitor cells in a first NK cell differentiation medium or second NK cell differentiation medium comprising at least one selected from the group consisting of:
β-mercaptoethanol (BME, 2BME, 2-ME or β-met), sodium selenite, ethanolamine, ascorbic acid, and IL-3.

6. The method according to claim 5, wherein the second NK cell differentiation medium does not contain IL-3.

7. The method according to claim 5, wherein the first NK cell differentiation medium or the second NK cell differentiation medium contains 0.1 to 20 μM β-mercaptoethanol, 0.1 to 50 ng/ml sodium selenite, 1 to 600 μM ethanolamine, and 1 to 200 mg/L ascorbic acid based on a total composition.

8. The method according to claim 5, wherein the first NK cell differentiation medium or the second NK cell differentiation medium further comprises at least one selected from the group consisting of stemline, stempro-34, SCF, IL-7, IL-15, and FLT3 ligand.

9. The method according to claim 5, wherein the NK cells have increased expression of an activating receptor.

10. The method according to claim 9, wherein the activating receptor is one or more of CD16 and NKG2D.

11. The method according to claim 5, wherein the second NK cell differentiation medium further comprises a p38 MAPK inhibitor.

12. The method according to claim 11, wherein the p38 MAPK inhibitor is one or more selected from the group consisting of adezmapimod (SB203580), losmapimod, doramapimod, ralimetinib, and neflamapimod (VX-745).

13. The method according to claim 11, wherein the NK cells have reduced expression of an inhibitory receptor.

14. The method according to claim 13, wherein the inhibitory receptor is NKG2A.

15. The method according to claim 14, wherein the NK cells have a maintained or increased expression level of one or more activating receptor selected from the group consisting of CD16, NKG2D, NKp30, and NKp44.

16. The method according to claim 5, wherein the culturing performed in the first NK cell differentiation medium or the second NK cell differentiation medium is performed from 10 to 14 days to 46 to 52 days based on an entire incubation process.

17. The method according to claim 16, wherein the culturing performed in the first NK cell differentiation medium is performed from 10 to 14 days to 16 to 20 days based on the entire incubation process.

18. The method according to claim 16, wherein the culturing performed in the second NK cell differentiation medium is performed from 16 to 20 days to 46 to 52 days based on the entire incubation process.

19. The method according to claim 11, wherein the NK cells have increased cytotoxicity.

20. The method according to claim 1, wherein the stem cells are one or more selected from the group consisting of induced pluripotent stem cells, embryonic stem cells, and adult stem cells.

21.-23. (canceled)
